SEQUENCE LISTING (4 sequences)

SEQ ID NO: 1 - protein, 6 aa, Spinacia oleracea
YHIEPV

SEQ ID NO: 2 - protein, 5 aa, Spinacia oleracea
YKLTY

SEQ ID NO: 3 - protein, 5 aa, Artificial Sequence
  synthesized peptide
YRLTY

SEQ ID NO: 4 - protein, 5 aa, Artificial Sequence
  synthesized peptide
YRETY

(19) United States
(12) Patent Application Publication
(10) Pub. No.: US 2024/0301001 A1
(43) Pub. Date: Sep. 12, 2024

(54) PEPTIDE-CONTAINING COMPOSITION

(71) Applicant: Kyoto University, Kyoto-shi, Kyoto (JP)

(72) Inventors: Kousaku OHINATA, Kyoto-shi, Kyoto (JP); Kentaro KANEKO, Kyoto-shi, Kyoto (JP); Yukihiro TAKEKUMA, Kyoto-shi, Kyoto (JP); Shimon ABE, Kyoto-shi, Kyoto (JP); Saya ARAKI, Kyoto-shi, Kyoto (JP)

(21) Appl. No.: 18/572,379

(22) PCT Filed: Jun. 24, 2022
(86) PCT No.: PCT/JP2022/025270

§ 371 Date: Dec. 20, 2023

(30) Foreign Application Priority Data
Jun. 24, 2021 (JP) 2021-104872

(51) Int. Cl. C07K 7/06; A23L 2/66; A23L 33/18; A61K 36/185; A61K 38/00; A61P 3/04

(52) U.S. Cl. CPC C07K 7/06; A23L 2/66; A23L 33/18; A61K 36/185; A61P 3/04; A61K 38/00

(57) ABSTRACT

Provided is a composition for improving leptin resistance, a composition for enhancing leptin sensitivity, a composition for suppressing food intake, or a composition for anti-obesity. Specifically, provided is a composition for improving leptin resistance or enhancing leptin sensitivity, comprising a peptide comprising the amino acid sequence YAB as an active ingredient, wherein A is an amino acid residue having a basic side chain, and B is an amino acid residue having a nonpolar side chain or an amino acid residue having an acidic side chain.

### TECHNICAL FIELD

The present disclosure relates to, for example, a peptide-containing composition.

### BACKGROUND ART

Leptin is secreted by adipocytes and is known as an anti-obesity hormone that acts mainly on the hypothalamus to suppress food intake and increase energy consumption.

In recent years, obesity has been on the rise due to, for example, the increased amount of fat intake. Typically, it is known that there is a positive correlation between fat accumulation level and leptin secretion level. In the case of obesity, “leptin resistance,” in which no body weight loss is observed even though the serum leptin level is often elevated, is known to be caused (Patent Literature (PTL) 1).

### CITATION LIST

### Patent Literature

PTL 1: JP2013-028560A
PTL 2: JP2020-509057A

### SUMMARY OF INVENTION

### Technical Problem

A problem to be solved by the present disclosure is to provide a composition for improving leptin resistance or a composition for enhancing leptin sensitivity. Another problem to be solved by the present disclosure is to provide a composition for suppressing food intake. Yet another problem to be solved by the present disclosure is to provide a composition for anti-obesity.

### Solution to Problem

The present inventors have previously discovered that a peptide comprising the amino acid sequence YHIEPV (SEQ ID NO: 1) has an anxiolytic effect (PTL 2). Typically, substances with an anxiolytic effect are known to often increase appetite; however, the present inventors discovered that a peptide comprising the amino acid sequence YHIEPV has an effect of suppressing high-fat diet intake, and they made further improvements.

The present disclosure encompasses, for example, the subject matter described in the following items.

### Item 1

A composition for improving leptin resistance or enhancing leptin sensitivity, comprising a peptide comprising the amino acid sequence YAB as an active ingredient,
wherein A is an amino acid residue having a basic side chain, and B is an amino acid residue having a nonpolar side chain or an amino acid residue having an acidic side chain.

### Item 2

The composition for improving leptin resistance or enhancing leptin sensitivity according to Item 1,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

### Item 3

A composition for improving leptin resistance or enhancing leptin sensitivity, comprising a pepsin-treated product or a pepsin-treated and pancreatin-treated product of Rubisco protein as an active ingredient.

### Item 4

A composition for suppressing food intake comprising a peptide comprising the amino acid sequence YAB as an active ingredient,
wherein A is an amino acid residue having a basic side chain, and B is an amino acid residue having a nonpolar side chain or an amino acid residue having an acidic side chain.

### Item 5

The composition for suppressing food intake according to Item 4,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

### Item 6

A composition for suppressing food intake comprising a pepsin-treated product or a pepsin-treated and pancreatin-treated product of Rubisco protein as an active ingredient.

### Item 7

A composition for anti-obesity comprising a peptide comprising the amino acid sequence YAB as an active ingredient,
wherein A is an amino acid residue having a basic side chain, and B is an amino acid residue having a nonpolar side chain or an amino acid residue having an acidic side chain.

### Item 8

The composition for anti-obesity according to Item 7,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

### Item 9

A composition for anti-obesity comprising a pepsin-treated product or a pepsin-treated and pancreatin-treated product of Rubisco protein as an active ingredient.

### Item 10

The composition according to any one of Items 1 to 9, which is an oral composition.

### Item 11

The composition according to any one of Items 1 to 10, which is a pharmaceutical composition.

### Item 12

The composition according to any one of Items 1 to 10, which is a food and drink composition.

### Item 13

A method for improving leptin resistance or enhancing leptin sensitivity, comprising
administering a peptide comprising the amino acid sequence YAB, wherein A is an amino acid residue having a basic side chain, and B is an amino acid residue having a nonpolar side chain or an amino acid residue having an acidic side chain, to a patient or a potential patient in need thereof.

### Item 14

A method for suppressing food intake, comprising
administering a peptide comprising the amino acid sequence YAB, wherein A is an amino acid residue having a basic side chain, and B is an amino acid residue having a nonpolar side chain or an amino acid residue having an acidic side chain, to a patient or a potential patient in need thereof.

### Item 15

An anti-obesity method, comprising
administering a peptide comprising the amino acid sequence YAB, wherein A is an amino acid residue having a basic side chain, and B is an amino acid residue having a nonpolar side chain or an amino acid residue having an acidic side chain, to a patient or a potential patient in need thereof.

### Item 16

The method according to any one of Items 13 to 15,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

### Item 17

A method for improving leptin resistance or enhancing leptin sensitivity, comprising
administering a pepsin-treated product or a pepsin-treated and pancreatin-treated product of Rubisco protein to a patient or a potential patient in need thereof.

### Item 18

A method for suppressing food intake, comprising
administering a pepsin-treated product or a pepsin-treated and pancreatin-treated product of Rubisco protein to a patient or a potential patient in need thereof.

### Item 19

An anti-obesity method, comprising
administering a pepsin-treated product or a pepsin-treated and pancreatin-treated product of Rubisco protein to a patient or a potential patient in need thereof.

### Item 20

Use of a peptide comprising the amino acid sequence YAB, wherein A is an amino acid residue having a basic side chain, and B is an amino acid residue having a nonpolar side chain or an amino acid residue having an acidic side chain, in the production of a medicinal drug or a food for improving leptin resistance or enhancing leptin sensitivity.

### Item 21

Use of a peptide comprising the amino acid sequence YAB, wherein A is an amino acid residue having a basic side chain, and B is an amino acid residue having a nonpolar side chain or an amino acid residue having an acidic side chain, in the production of a medicinal drug or a food for suppressing food intake.

### Item 22

Use of a peptide comprising the amino acid sequence YAB, wherein A is an amino acid residue having a basic side chain, and B is an amino acid residue having a nonpolar side chain or an amino acid residue having an acidic side chain, in the production of a medicinal drug or a food for suppressing obesity.

### Item 23

The use according to any one of Items 20 to 22,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

### Item 24

Use of a pepsin-treated product or a pepsin-treated and pancreatin-treated product of Rubisco protein in the production of a medicinal drug or a food for improving leptin resistance or enhancing leptin sensitivity.

### Item 25

Use of a pepsin-treated product or a pepsin-treated and pancreatin-treated product of Rubisco protein in the production of a medicinal drug or a food for suppressing food intake.

### Item 26

Use of a pepsin-treated product or a pepsin-treated and pancreatin-treated product of Rubisco protein in the production of a medicinal drug or a food for suppressing obesity.

### Advantageous Effects of Invention

For example, a composition for improving leptin resistance or a composition for enhancing leptin sensitivity is provided. Further, for example, a composition for suppressing food intake is provided. Furthermore, for example, a composition for anti-obesity is provided.

### BRIEF DESCRIPTION OF DRAWINGS

FIG. 1 shows the results obtained by evaluating the impact of green leaf Rubisco digested product A or B on high-fat diet intake.

FIG. 2 shows the results obtained by evaluating the impact of oral administration of Rubisco-derived peptide YHIEPV on high-fat diet intake.

FIG. 3 shows the results obtained by evaluating the impact of an antagonist on high-fat diet intake suppression induced by oral administration of YHIEPV.

FIG. 4 shows the results obtained by evaluating the impact of long-term oral administration of YHIEPV on body weight gain caused by a high-fat diet.

FIG. 5 shows the results obtained by evaluating the impact of five-week oral administration of YHIEPV on blood leptin level and fat mass.

FIG. 6 shows the results obtained by evaluating the impact of YHIEPV on intracellular leptin sensitivity in the hypothalamus.

FIG. 7 shows the results obtained by evaluating the impact of YHIEPV on leptin resistance induced by Forskolin.

FIG. 8 shows the results obtained by evaluating the impact of oral administration of YHIEPV on the anti-obesity effect of intracerebroventricularly administered leptin.

FIG. 9 shows the results obtained by evaluating the impact of oral administration of YHIEPV on stress response in the brain caused by high-fat diet intake.

FIG. 10 shows the results obtained by evaluating the impact of oral administration of Rubisco-derived peptide YKLTY on high-fat diet intake.

FIG. 11 shows the results obtained by evaluating the impact of YKLTY on intracellular leptin sensitivity in the hypothalamus.

FIG. 12 shows the results obtained by evaluating the impact of oral administration of peptides from spinach, algae, and bacteria Rubisco on high-fat diet intake.

### DESCRIPTION OF EMBODIMENTS

Embodiments encompassed by the present disclosure are described in more detail below.

The compositions encompassed by the present disclosure preferably comprise a peptide comprising the amino acid sequence YAB. In the present specification, such compositions may be referred to as “the composition of the present disclosure.” The composition of the present disclosure more preferably comprises a peptide comprising the amino acid sequence YAB as an active ingredient.

In the amino acid sequence YAB, A is preferably an amino acid residue having a basic side chain. Examples of amino acid residues having a basic side chain include lysine, arginine, and histidine. In particular, lysine, arginine, or histidine is preferred.

In the amino acid sequence YAB, B is preferably an amino acid residue having a nonpolar side chain or an amino acid residue having an acidic side chain. Examples of amino acid residues having a nonpolar side chain include alanine, valine, leucine, isoleucine, proline, phenylalanine, methionine, and tryptophan. In particular, leucine or isoleucine is preferred, and leucine is more preferred. Examples of amino acid residues having an acidic side chain include glutamic acid and aspartic acid. In particular, glutamic acid is preferred.

In particular, in the amino acid sequence YAB, it is preferable that A is an amino acid residue having a basic side chain, and that B is an amino acid residue having a nonpolar side chain or an amino acid residue having an acidic side chain. The amino acid residue having a basic side chain is preferably lysine, arginine, or histidine. The amino acid residue having a nonpolar side chain is preferably leucine or isoleucine, and more preferably leucine. The amino acid residue having an acidic side chain is preferably glutamic acid. When A as the amino acid residue having a basic side chain is histidine, B is more preferably isoleucine, which is an amino acid residue having a nonpolar side chain. When A as the amino acid residue having a basic side chain is lysine, B is more preferably leucine, which is an amino acid residue having a nonpolar side chain. When A as the amino acid residue having a basic side chain is arginine, B is more preferably leucine, which is an amino acid residue having a nonpolar side chain, or glutamic acid, which is an amino acid residue having an acidic side chain.

The number of amino acids constituting the peptide comprising the amino acid sequence YAB may be, for example, 3 to 25 or may be 3 to 10. In particular, the number is preferably 5 or 6.

In other words, additional amino acid residues may be further added to the amino acid sequence YAB. The number of amino acid residues to be added may be, for example, 1 to 22 or may be 1 to 7. In particular, the number is preferably 2 or 3.

The amino acid residues to be added are not particularly limited, and any amino acid residue can be added.

The amino acid residue to be added may be added to the N-terminal side or the C-terminal side of the amino acid sequence YAB. In particular, the addition is preferably made to the C-terminal side of the amino acid sequence YAB.

More specific examples of the peptide comprising the amino acid sequence YAB include a peptide comprising YHIEPV (SEQ ID NO: 1), a peptide comprising YKLTY (SEQ ID NO: 2), a peptide comprising YRLTY (SEQ ID NO: 3), and a peptide comprising YRETY (SEQ ID NO: 4). The peptides comprising the amino acid sequence YAB can be used alone or in a combination of two or more.

The peptide comprising the amino acid sequence YAB may also be, for example, a peptide comprising an amino acid sequence in which at least one amino acid is deleted, substituted, or added in the amino acid sequence represented by SEQ ID NO: 1, 2, 3, or 4.

The number of amino acids deleted, substituted, or added in the amino acid sequence in which at least one amino acid is deleted, substituted, or added in the amino acid sequence represented by SEQ ID NO: 1, 2, 3, or 4 may be, for example, 1, 2, 3, 4, 5, 6, 7, 8, 9, 10, 11, 12, 13, 14, 15, 16, 17, 18, 19, or 20. For example, the number may be 1 to 20, 1 to 10, 1 to 5, or 1 to 3.

When an amino acid is added in the amino acid sequence represented by SEQ ID NO: 1, 2, 3, or 4, the addition may be made to the N-terminal side or the C-terminal side of the amino acid sequence represented by SEQ ID NO: 1, 2, 3, or 4. In particular, the addition is preferably made to the C-terminal side of the amino acid sequence represented by SEQ ID NO: 1, 2, 3, or 4.

When an amino acid is substituted in the amino acid sequence represented by SEQ ID NO: 1, 2, 3, or 4, the substitution is preferably a conservative substitution. In the present specification, “conservative substitution” means a substitution of a specific amino acid with an amino acid having a side chain with properties similar to the side chain of the specific amino acid. Specific examples of conservative substitutions include a substitution between amino acid residues having a basic side chain, such as lysine, arginine, and histidine; a substitution between amino acid residues having an acidic side chain, such as aspartic acid and glutamic acid; a substitution between amino acid residues having an uncharged polar side chain, such as glycine, asparagine, glutamine, serine, threonine, tyrosine, and cysteine; a substitution between amino acid residues having a nonpolar side chain, such as alanine, valine, leucine, isoleucine, proline, phenylalanine, methionine, and tryptophan; a substitution between amino acid residues having a β-branched side chain, such as threonine, valine, and isoleucine; and a substitution between amino acid residues having an aromatic side chain, such as tyrosine, phenylalanine, tryptophan, and histidine.

Techniques for adding a mutation, such as deletion, substitution, or addition of at least one amino acid, to a specific amino acid sequence are well known in the related technical fields, and any method can be used. For example, restriction enzyme treatment, treatment with exonuclease, DNA ligase, etc., site-specific mutagenesis, and random mutagenesis may be used.

The amino acid residues that constitute the peptide comprising the amino acid sequence YAB can include natural and/or non-natural amino acid residues, unless otherwise specified. Examples of natural amino acids include amino acid residues that constitute a protein, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; and other amino acid residues, such as selenocysteine, N-formylmethionine, pyrrolysine, and pyroglutamine. Examples of non-natural amino acids include azetidinecarboxylic acid, 2-aminoadipic acid, 3-aminoadipic acid, β-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tert-butylglycine, 2,4-diaminoisobutyric acid, desmosine, 2,2-diaminopimelic acid, 2,3-diaminopropionic acid, N-ethylglycine, N-ethylasparagine, homoproline, hydroxylysine, allohydroxylysine, 3-hydroxyproline, 4-hydroxyproline, isodesmosine, alloisoleucine, N-methylalanine, N-methylglycine, N-methylisoleucine, N-methylpentylglycine, N-methylvaline, naphthalanine, norvaline, norleucine, ornithine, pentylglycine, pipecolinic acid, and thioproline.

Any of L-, D-, and DL-amino acids (including any of racemic mixtures and amino acids with an excess of one of the enantiomers insofar that they are mixtures of D- and L-amino acids) can be used as the amino acids constituting the peptide. In particular, peptides consisting of L-amino acids or consisting of D-amino acids are preferred.

If the peptide comprising the amino acid sequence YAB contains two or more asymmetric carbon atoms, the peptide may be present in the form of enantiomers or diastereomers in any ratio in any form. Enantiomers and diastereomers can be separated by using a commonly used column. Any known method can be used for separation, such as a method that uses an optically active column; a method comprising carrying out optical resolution with an introduced optically active group, followed by removal of the optically active group; and a method comprising forming an optically active salt with an acid or base, followed by performing optical resolution.

The peptide comprising the amino acid sequence YAB may be in the form of a salt (acid addition salt or basic salt). Examples of acid addition salts include inorganic salts, such as hydrochloride, sulfate, nitrate, phosphate, hydrobromide, and perchlorate; and salts of organic acids, such as citric acid, succinic acid, maleic acid, fumaric acid, malic acid, tartaric acid, p-toluenesulfonic acid, benzenesulfonic acid, methanesulfonic acid, and trifluoroacetic acid. Examples of basic salts include salts of alkali metals, such as sodium, potassium, and lithium; and salts of alkaline earth metals, such as calcium and magnesium. Examples of bases that can be used to produce base addition salts include sodium hydroxide, potassium hydroxide, lithium hydroxide, and calcium hydroxide. Additional acids and bases that can produce pharmaceutically acceptable salts are described in Stahl and Wermuth, Eds., 2008, Handbook of Pharmaceutical Salts: Properties, Selection and Use, Verlag Helvetica Chimica Acta, Zurich, Switzerland.

The peptide comprising the amino acid sequence YAB may be in the form of a solvate. Examples of solvates include those with water (for hydrates), methanol, ethanol, isopropanol, acetic acid, tetrahydrofuran, acetone, dimethylformamide, dimethylsulfoxide, dimethylacetamide, acetamide, ethylene glycol, propylene glycol, and dimethoxyethane.

The peptide comprising the amino acid sequence YAB may also be synthesized by using any known peptide synthesis method, enzymatic method, and the like. The peptide may also be obtained from microorganisms or cultured cells that have been engineered to produce the peptide comprising the amino acid sequence YAB; for example, the peptide may be obtained from microorganisms or cultured cells in which a gene encoding the peptide has been inserted. The peptide may otherwise be obtained by in vitro translation.

The content of the peptide comprising the amino acid sequence YAB in the composition of the present disclosure is not particularly limited and can be suitably set up to 100% by mass.

The composition of the present disclosure may also comprise a pepsin-treated product of Rubisco (ribulose 1,5-bisphosphate carboxylase/oxygenase) protein. The composition of the present disclosure more preferably comprises a pepsin-treated product of Rubisco protein as an active ingredient.

A peptide comprising YKLTY (SEQ ID NO: 2) is contained in a pepsin-treated product of Rubisco protein as demonstrated in the Examples below. Further, a peptide comprising YRLTY (SEQ ID NO: 3) and a peptide comprising YRETY (SEQ ID NO: 4) are predicted to be contained in a pepsin-treated product of Rubisco protein.

Further, the composition of the present disclosure may comprise a pepsin-treated and pancreatin-treated product of Rubisco (ribulose 1,5-bisphosphate carboxylase/oxygenase) protein. The composition of the present disclosure more preferably comprises a pepsin-treated and pancreatin-treated product of Rubisco protein as an active ingredient.

A peptide comprising YHIEPV (SEQ ID NO: 1) is contained in a pepsin-treated and pancreatin-treated product of Rubisco protein as demonstrated in the Example below.

These can be used alone or in a combination of two or more.

Rubisco protein is a protein involved in carbon dioxide fixation in green leaves. Rubisco protein is abundant in plants and is considered the most abundant protein on earth. Green plants containing Rubisco protein include plants with edible green leaf portions, such as kale, young barley leaves, young wheat leaves, ashitaba, young mulberry leaves, spinach, mulukhiya, cabbage, and tea. In particular, spinach is preferred.

Rubisco protein is also known to be synthesized by algae, bacteria, and the like. Examples of algae that synthesize Rubisco protein include euglena and chlorella. Examples of bacteria that synthesize Rubisco protein include hydrogen-oxidizing bacteria and purple non-sulfur bacteria.

In particular, the source of Rubisco protein for use in the present disclosure is preferably spinach, algae (more specifically, euglena, chlorella, etc.), and bacteria (more specifically, hydrogen-oxidizing bacteria, purple non-sulfur bacteria, etc.).

Pepsin (EC. 3.4.23.1-3) is a type of protease that functions in the stomach of animals. Pepsin can be used as a food additive in Japan. Commercially available products, such as pepsin (reagent grade) and pepsin (food additive grade), can be used.

Pancreatin is a mixture of enzymes secreted from the pancreas and contains lipase, amylase, protease (trypsin, chymotrypsin, etc.), and the like. Pancreatin can be used as a food additive in Japan. Commercially available products, such as pancreatin (reagent grade) and pancreatin (food additive grade), can be used.

The substrate to be treated with pepsin or with pepsin and pancreatin is not particularly limited as long as the substrate contains Rubisco protein. Examples include green plants themselves, algae themselves, and bacteria themselves; squeezed juices of green plants, algae, or bacteria (e.g., green juices); and purified Rubisco protein.

Rubisco protein is easily extracted as a soluble protein from spinach, algae (more specifically, euglena, chlorella, etc.), bacteria (more specifically, hydrogen-oxidizing bacteria, purple non-sulfur bacteria, etc.), and the like.

The treatment temperature with pepsin or with pepsin and pancreatin is suitably selected from, for example, 30 to 70° C., 30 to 40° C., 40 to 70° C., and 50 to 65° C.

The treatment time is suitably selected from, for example, about 30 minutes to 48 hours, about 1 to 10 hours, and about 2 to 8 hours.

The pH during treatment with pepsin can be suitably selected from, for example, a pH of about 1.5 to 3.5, and preferably a pH of about 2 to 3. The pH during treatment with pancreatin can be suitably selected from, for example, a pH of about 6.5 to 8.5, and preferably a pH of about 7 to 8.

Since pepsin and pancreatin have different optimum pH values of enzyme, the treatment with pepsin and the treatment with pancreatin are preferably performed separately. In this case, the treatment with pepsin and the treatment with pancreatin may be performed in any order. For example, the treatment with pancreatin may be performed after the treatment with pepsin, and the treatment with pepsin may be performed after the treatment with pancreatin.

Pepsin and pancreatin may be inactivated if necessary by heating them to a temperature that allows inactivation of pepsin and pancreatin (e.g., heating at a temperature exceeding 80° C. for about 5 to 60 minutes).

The pepsin-treated product and the pepsin-treated and pancreatin-treated product of Rubisco protein may be optionally purified. Purification methods are known in the related technical fields, and any method can be used.

The content of the pepsin-treated product and the pepsin-treated and pancreatin-treated product of Rubisco protein in the composition of the present disclosure is not particularly limited and can be suitably set up to 100% by mass.

The composition of the present disclosure comprises the peptide comprising the amino acid sequence YAB, and the pepsin-treated product of Rubisco protein and/or the pepsin-treated and pancreatin-treated product of Rubisco protein, and may further comprise other components. Examples of the other components include components and materials that can be used as other drugs and/or other foods, such as pharmaceutically or food-hygienically acceptable bases, carriers, solvents, dispersants, emulsifiers, buffers, stabilizers, excipients, binders, disintegrants, lubricants, thickeners, antioxidants, preservatives, coating agents, colorants, and gastric mucosal protectants. These components can be used alone or in a combination of two or more.

The form of the composition of the present disclosure is not particularly limited. Examples include tablets, pills, capsules, powders, fine granules, granules, liquids, troches, jelly, injections, plasters, extracts, suppositories, suspensions, tinctures, ointments, poultices, nasal drops, inhalants, liniments, lotions, and aerosols.

The composition of the present disclosure can be prepared by using common methods by combining the peptide comprising the amino acid sequence YAB, the pepsin-treated product of Rubisco protein and/or the pepsin-treated and pancreatin-treated product of Rubisco protein, and optionally other components.

The composition of the present disclosure is preferably an oral composition. Specific examples include pharmaceutical compositions and food and drink compositions. The food and drink composition may be, for example, a functional food, a dietary supplement, a supplement, a food for health use, a food for specified health use, a food with nutrient function claims, a food with functional claims, and the like.

Examples of the food and drink composition include drinks (coffee, hot chocolate, juices, soft drinks, mineral drinks, tea drinks, green tea, black tea, oolong tea, milk drinks, lactic acid bacteria drinks, yogurt drinks, carbonated drinks, other non-alcoholic drinks, alcohol drinks, etc.), snacks (Japanese hard candies, gums, gummies, gelatin desserts, puddings, mousses, cakes, candies, cookies, crackers, biscuits, chocolate, iced sweets (ice cream, ice pops, sherbet, ice shavings, etc.), etc.), furikake toppings, dressings, seasonings, processed meat foods (hamburger patties, meatloaf, meatballs, tsukune (grilled chicken meatball), etc.), processed fish foods (kamaboko (steamed fish paste), chikuwa (fish sausage), etc.), processed retort foods, and jelly-like foods (jellies, agars, jelly-like drinks, etc.). The food and drink composition may be a food prepared using a green plant as a raw material, such as powdered green tea, green juice, and vegetable juice.

As demonstrated in the Examples described later, the composition of the present disclosure exhibits, for example, an effect of suppressing high-fat diet intake, an effect of reducing body weight, an effect of enhancing leptin sensitivity, and/or an effect of improving leptin resistance. Therefore, the composition of the present disclosure can be suitably used, for example, for suppressing food intake, for anti-obesity, for enhancing leptin sensitivity, and/or for improving leptin resistance.

In the present specification, “leptin resistance” means a state in which the activation pathway response to leptin stimulation (e.g., phosphorylation of STAT3) is decreased at the cellular level, and means a state in which the effect of suppressing food intake and/or the effect of suppressing body weight gain of leptin is decreased at the individual level.

Without wishing to be bound by theory, it is believed that the composition of the present disclosure acts via a receptor involved in feeding behavior, i.e., δ opioid receptor or MC₄ receptor.

Also, without wishing to be bound by theory, it is believed that the composition of the present disclosure enhances leptin sensitivity by suppressing the Rap1 activation pathway.

Examples of the administration methods of the composition of the present disclosure include oral administration and parenteral administration (e.g., intravenous, arterial, intramuscular, subcutaneous, intraperitoneal, rectal, transdermal, and topical administration). In particular, oral administration is preferred.

Examples of the subject for administration (intake) of the composition of the present disclosure include, but are not particularly limited to, humans and non-human mammals (e.g., rats, mice, rabbits, cows, pigs, dogs, cats, sheep, and monkeys). Examples of human subjects include humans who are obese or suspected of being obese, humans who have leptin resistance or are suspected of having leptin resistance, humans who need to suppress the amount of food intake, humans who need to reduce their body weight, and humans who have two or more of these characteristics.

Examples of non-human mammals include mammals kept as pets, livestock, experimental animals, and the like. Examples of such non-human mammals include dogs, cats, monkeys, cows, horses, sheep, goats, pigs, rabbits, mice, rats, camels, and llamas.

The amount of administration (intake) of the composition of the present disclosure is not particularly limited and is determined depending on, for example, the age, body weight, and gender of the subject to be administered, the administration method, and the type of active ingredient.

For example, when the active ingredient is a peptide comprising the amino acid sequence YAB, the administration amount of the active ingredient is, for example, 0.01 mg/kg to 500 mg/kg, preferably 0.05 mg/kg to 100 mg/kg, and more preferably 0.1 to 30 mg/kg, per day for adults.

For example, when the active ingredient is a pepsin-treated product of Rubisco protein, the administration amount of the active ingredient is, for example, 1 mg/kg to 50000 mg/kg, preferably 5 mg/kg to 10000 mg/kg, and more preferably 10 to 3000 mg/kg, per day for adults.

For example, when the active ingredient is a pepsin-treated and pancreatin-treated product of Rubisco protein, the administration amount of the active ingredient is, for example, 1 mg/kg to 50000 mg/kg, preferably 5 mg/kg to 10000 mg/kg, and more preferably 10 to 3000 mg/kg, per day for adults.

The administration (intake) of the composition of the present disclosure may be performed once a day or multiple times per day (for example, about 2 to 5 times) by dividing it into multiple portions.

In the present specification, the terms “comprising,” “containing,” and “having” also include “consisting essentially of” and “consisting of.” The present disclosure encompasses any combination of the elements described in the present specification.

The various characteristics (e.g., properties, structures, and functions) described in each of the above embodiments of the present disclosure may be combined in any way to specify the subject matter encompassed by the present disclosure. In other words, the present disclosure encompasses any subject matter comprising all combinations of the combinable characteristics described in the present specification.

### EXAMPLES

The details of the present disclosure are specifically described with reference to the following Examples. However, the present disclosure is not limited to the Examples. Below, experiments were conducted at an ordinary temperature under atmospheric pressure unless otherwise specified. Further, “%” means “% by mass” unless otherwise specified.

### Production Example 1

### Extraction of Rubisco Protein

Spinach was homogenized. The pH of the resulting homogenate was adjusted to a pH of 11 with a 1 N aqueous NaOH solution. The resulting homogenate was filtered through two layers of gauze. The filtrate was centrifuged at 13,500 G and 5° C. for 50 minutes. After centrifugation, the supernatant was filtered. The pH of the resulting filtrate was adjusted to a pH of 4.5 with acetic acid to precipitate Rubisco protein. The precipitate was washed with acetone, ethanol, and diethyl ether, and dried in a vacuum chamber to obtain a Rubisco powder.

### Green Leaf Rubisco Digested Product A

An enzyme digestion product of purified Rubisco protein was prepared under the following conditions (pepsin+pancreatin digestion).

Enzyme treatments were performed in the order of 1) and 2).
1) Pepsin (Sigma-Aldrich Co., LLC):Rubisco=1:100 (weight ratio, final concentration of Rubisco: 0.99 mg/ml); reaction temperature: 37° C.; reaction time: 5 hours; and reaction buffer: pH of 2.0.
2) Pancreatin (Sigma-Aldrich Co., LLC):Rubisco=1:20 (weight ratio, final concentration of Rubisco: 0.86 mg/ml); reaction temperature: 37° C.; reaction time: 5 hours; and reaction buffer: pH of 7.5.

After the reaction time stated above elapsed, the sample was boiled (100° C. for 10 minutes) to end the enzyme reaction.

### Green Leaf Rubisco Digested Product B

An enzyme digestion product of purified Rubisco protein was prepared under the following conditions (pepsin digestion).

Enzyme treatment was performed.
1) Pepsin (Sigma-Aldrich Co., LLC): Rubisco=1:100 (weight ratio, final concentration of Rubisco: 0.99 mg/ml); reaction temperature: 37° C.; reaction time: 5 hours; and reaction buffer: pH of 2.0.

### Production Example 2

### Peptide

YHIEPV (SEQ ID NO: 1), YKLTY (SEQ ID NO: 2), YRLTY (SEQ ID NO: 3), and YRETY (SEQ ID NO: 4) were synthesized by using a standard method, typically by a solid-phase method using the Fmoc strategy.

It was confirmed that YHIEPV (SEQ ID NO: 1) was contained in the green leaf Rubisco digested product A and YKLTY (SEQ ID NO: 2) was contained in the green leaf Rubisco digested product B.

According to the ortholog analysis of Rubisco protein, YRLTY (SEQ ID NO: 3) and YRETY (SEQ ID NO: 4) are peptides predicted to be contained in pepsin digestion products of Rubisco protein in algae (for example, euglena and chlorella) and in bacteria (for example, hydrogen-oxidizing bacteria and purple non-sulfur bacteria), respectively.

### Test Example 1: High-Fat Diet Intake with Green Leaf Rubisco Digested Product A or B

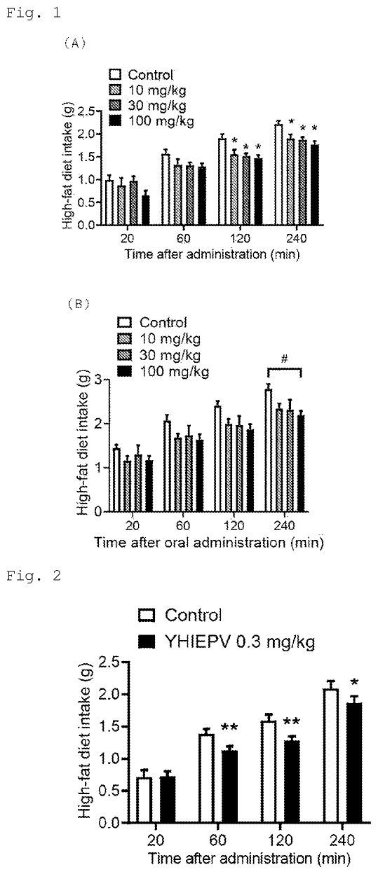

Male mice raised on a high-fat diet (60% kcal fat) were orally administered with the digested product under overnight fasting conditions, and the amount of food intake was measured over time (N=14-15). FIG. 1(A) shows the results. The data were analyzed by two-way analysis of variance, followed by multiple comparisons by Tukey's test. In FIG. 1(A), “*” indicates that p<0.05. The error bars represent standard error (n=14-15).

As shown in FIG. 1(A), it was confirmed that the green leaf Rubisco digested product A suppressed high-fat diet intake.

A test was performed in the same manner using the green leaf Rubisco digested product B instead of the green leaf Rubisco digested product A. FIG. 1(B) shows the results. The data were analyzed by two-way analysis of variance, followed by multiple comparisons by Tukey's test. In FIG. 1(B), “#” indicates that p<0.1. The error bars represent standard error (n=9).

As shown in FIG. 1(B), it was confirmed that the green leaf Rubisco digested product B suppressed high-fat diet intake.

### Test Example 2: High-fat Diet Intake with Oral Administration of Rubisco-Derived Peptide YHIEPV

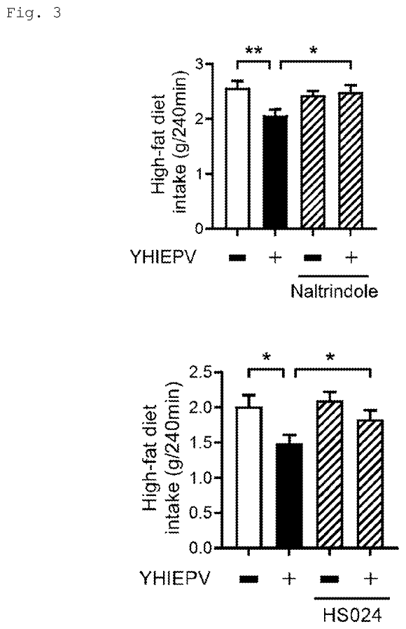

Male mice raised on a high-fat diet (60% kcal fat) were orally administered with Rubisco-derived peptide YHIEPV under overnight fasting conditions, and the amount of food intake was measured over time (N=11). FIG. 2 shows the results. The data were analyzed by two-way analysis of variance, followed by multiple comparisons by the Bonferroni test. In FIG. 2, “*” indicates that p<0.05. The error bars represent standard error (n=11).

As shown in FIG. 2, it was confirmed that Rubisco-derived peptide YHIEPV suppressed high-fat diet intake.

### Test Example 3: Impact of Antagonist on High-Fat Diet Intake Suppression Induced by Oral Administration of YHIEPV

Male mice raised on a high-fat diet (60% kcal fat) were administered under overnight fasting conditions with YHIEPV (oral administration) and naltrindole (intraperitoneal administration), which is a δ opioid receptor inhibitor, or HS024 (intracerebroventricular administration), which is an MC₄ receptor inhibitor, and the amount of food intake was measured 4 hours later (N=13-15). The δ opioid receptor and MC₄ receptor are receptors involved in feeding behavior. FIG. 3 shows the results. The data were analyzed by one-way analysis of variance, followed by multiple comparisons by Tukey's test. In FIG. 3, “*” indicates that p<0.05, and “*” indicates that p<0.01. The error bars represent standard error (n=13-15).

As shown in FIG. 3, it was confirmed that the effect of suppressing high-fat diet intake induced by oral administration of YHIEPV was inhibited by the δ-opioid receptor inhibitor or the MC₄ receptor inhibitor. This suggests that YHIEPV acts via these receptors.

### Test Example 4: Impact of Long-Term Oral Administration of YHIEPV on Body Weight Gain Caused by High-Fat Diet

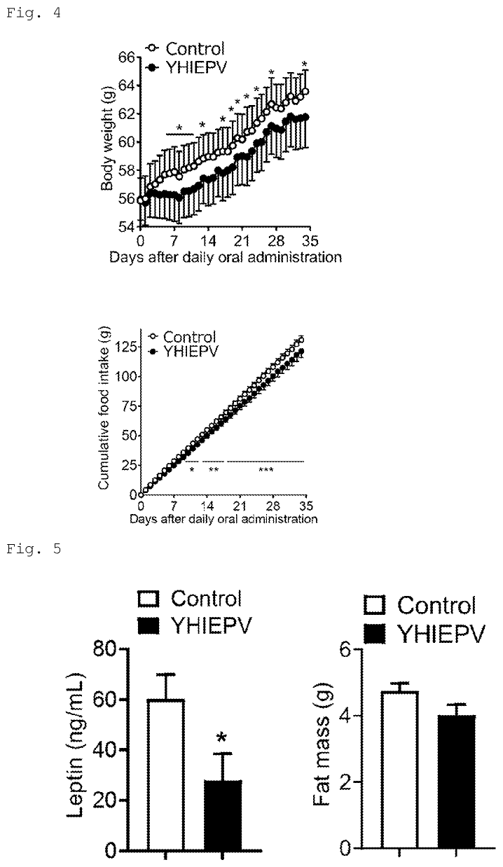

Male mice raised for at least one month on a high-fat diet (60% kcal fat) were orally administered with YHIEPV (0.3 mg/kg) once a day. The body weight and the amount of food intake were measured daily, and the amount of food intake was calculated as cumulative food intake (N=10). FIG. 4 shows the results. The data were analyzed by two-way analysis of variance, followed by multiple comparisons by the Bonferroni test. In FIG. 4, “*” indicates that p<0.05, “**” indicates that p<0.01, and “***” indicates that p<0.001. The error bars represent standard error (n=10).

As shown in FIG. 4, it was confirmed that the long-term oral administration of YHIEPV suppressed body weight gain caused by a high-fat diet.

### Test Example 5: Blood Leptin Level after Five-Week Oral Administration of YHIEPV

After the five-week oral administration of YHIEPV in Test Example 4, the blood was collected under anesthesia. Subsequently, the blood was centrifuged, and the serum was collected. The leptin level in the serum was determined by ELISA (according to the manufacturer's protocol) (N=10). The fat mass (peritesticular fat and mesenteric fat) was also measured at the same time as blood sampling (N=9). FIG. 5 shows the results. The data were analyzed by t-test. In FIG. 5, “*” indicates that p<0.05. The error bars represent standard error (n=9-10).

As shown in FIG. 5, it was confirmed that the five-week oral administration of YHIEPV decreased the blood leptin level. The fat mass showed a trend toward decrease.

### Test Example 6: YHIEPV-Induced Intracellular Leptin Sensitivity in the Hypothalamus

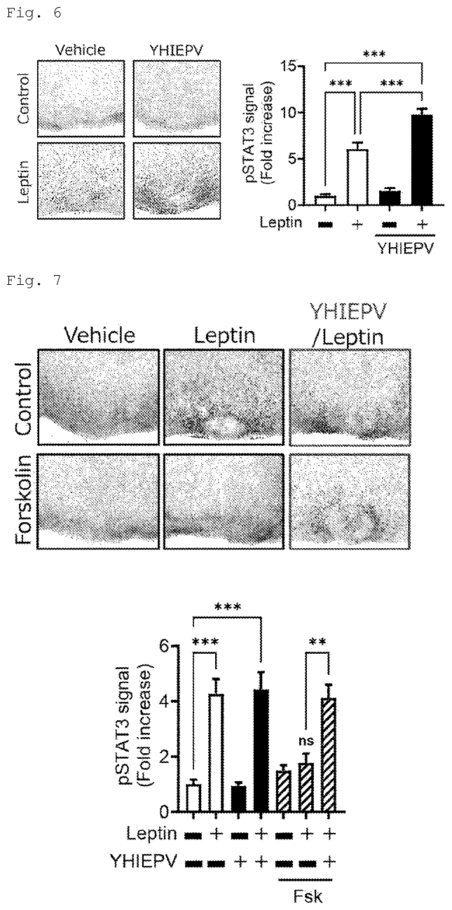

A hypothalamic organ culture system (Kaneko et al., Cell Reports 2016) was preincubated with YHIEPV (100 μM) for 24 hours, followed by leptin stimulation (30 nM) for 1 hour. Subsequently, formalin fixation was performed and the phosphorylated STAT3 signal was detected by an immunostaining chemistry method (N=6-7). FIG. 6 shows the results. The data were analyzed by one-way analysis of variance, followed by multiple comparisons by Tukey's test. In FIG. 6, “***” indicates that p<0.001. The error bars represent standard error (n=6-8).

As shown in FIG. 6, it was confirmed that YHIEPV enhanced intracellular leptin sensitivity in the hypothalamus.

### Test Example 7: Impact of YHIEPV on Leptin Resistance Induced by Forskolin (Rap1 Pathway Stimulation)

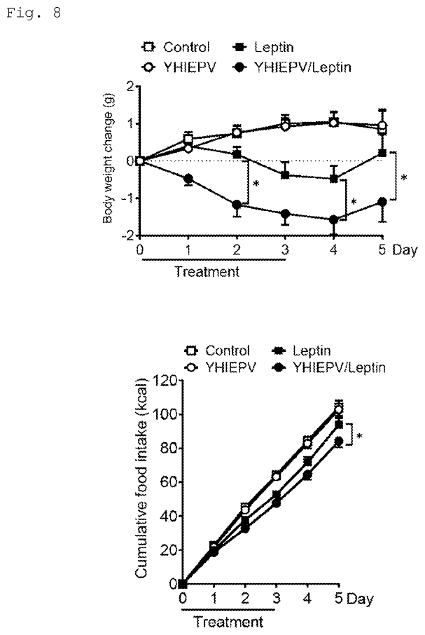

A hypothalamic organ culture system was preincubated with Forskolin (20 μM) and YHIEPV (100 μM) for 6 hours, followed by leptin stimulation (60 nM) for 1 hour. Subsequently, formalin fixation was performed and the phosphorylated STAT3 signal was detected by an immunostaining chemistry method (N=6-7). Forskolin is known to induce leptin resistance via the Rap1 activation pathway. FIG. 7 shows the results. The data were analyzed by one-way analysis of variance, followed by multiple comparisons by Tukey's test. In FIG. 7, “**” indicates that p<0.01, and “***” indicates that p<0.001. The error bars represent standard error (n=5-9).

As shown in FIG. 7, it was confirmed that leptin resistance induced by Forskolin was improved by YHIEPV. This suggests that YHIEPV enhances leptin sensitivity by suppressing the Rap1 activation pathway.

### Test Example 8: Impact of Oral Administration of YHIEPV on the Anti-obesity Effect of Intracerebroventricularly Administered Leptin

A guide cannula was placed in the lateral ventricle of male mice raised on a high-fat diet (60% kcal fat) for at least one month, and a recovery period of at least 1 week was then provided. Subsequently, the body weight change and the amount of food intake were measured after the addition of YHIEPV (0.3 mg/kg, oral administration) and leptin (0.5 μg, intracerebroventricular administration) (N=10-12). FIG. 8 shows the results. The data were analyzed by two-way analysis of variance, followed by multiple comparisons by Tukey's test. In FIG. 7, “*” indicates that p<0.05. The error bars represent standard error (n=10-12).

As shown in FIG. 8, it was confirmed that the anti-obesity effect of the intracerebroventricularly administered leptin was enhanced by oral administration of YHIEPV.

### Test Example 9: Impact of Oral Administration of YHIEPV on Stress Response in the Brain Caused by High-Fat Diet Intake

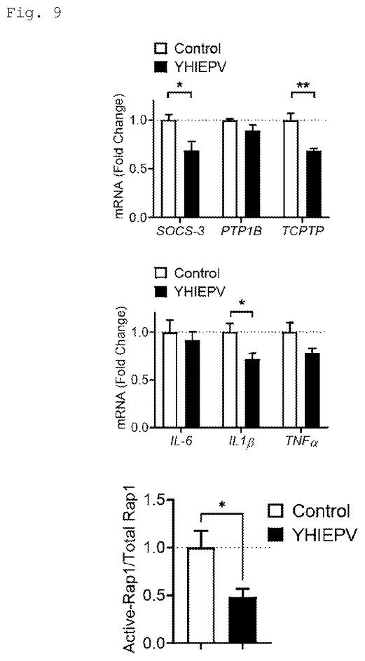

Male mice were raised for at least one month on a high-fat diet (60% kcal fat) and were orally administered with YHIEPV (0.3 mg/kg) for 5 days, and hypothalamic and brain samples were then collected. The expression levels of genes associated with leptin resistance (SOCS-3, PTP1B, and TCPTP) and inflammatory cytokines (IL-6, IL1β, TNFα) in the hypothalamus were measured by RT-qPCR. Active Rap1 in the brain was quantified by using the Active Rap1 Pull-down assay kit and the Detection Kit according to the manufacturer's protocol (N=4). FIG. 9 shows the results. The data were analyzed by t-test. In FIG. 9, “*” indicates that p<0.05. The error bars represent standard error (n=4).

As shown in FIG. 9, it was confirmed that the oral administration of YHIEPV significantly decreased the expression levels of SOCS-3 and TCPTP, and showed a decreasing trend in the expression level of PTP1B. It was also confirmed that the oral administration of YHIEPV significantly decreased the expression level of IL1β and showed a decreasing trend in the expression levels of IL-6 and TNFα. It was also confirmed that the oral administration of YHIEPV significantly decreased the expression level of active Rap1. These results suggest that YHIEPV enhances leptin sensitivity by suppressing the stress response and Rap1 activation pathway.

### Test Example 10: High-Fat Diet Intake with Oral Administration of Rubisco-Derived Peptide YKLTY

The amount of food intake was measured in the same manner as in Test Example 2, except that YKLTY was used instead of YHIEPV (N=7-8). FIG. 10 shows the results. The data were analyzed by two-way analysis of variance, followed by multiple comparisons by Tukey's test. In FIG. 10, “*” indicates that p<0.05. The error bars represent standard error (n=7-8).

As shown in FIG. 10, it was confirmed that Rubisco-derived peptide YKLTY suppressed high-fat diet intake.

### Test Example 11: YKLTY-Induced Intracellular Leptin Sensitivity in the Hypothalamus

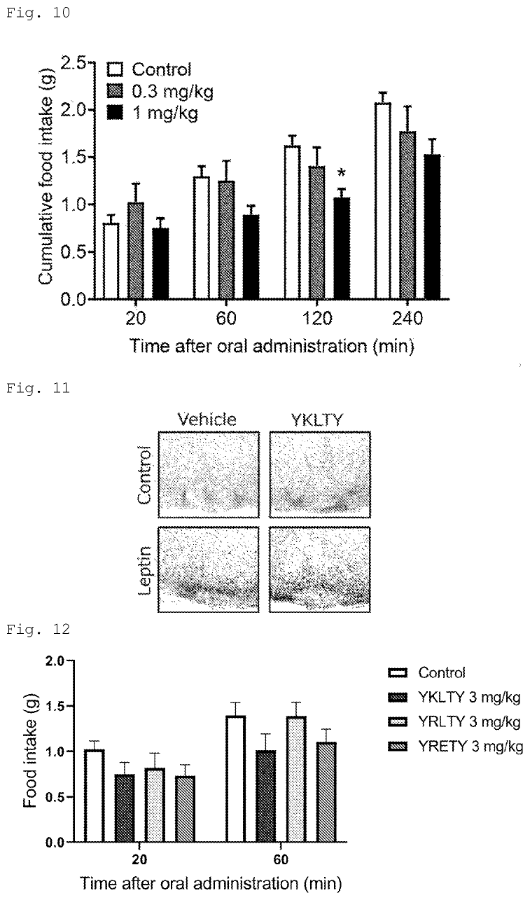

Leptin sensitivity was evaluated in the same manner as in Test Example 6, except that YKLTY was used instead of YHIEPV (N=6-7). FIG. 11 shows the results.

As shown in FIG. 11, it was confirmed that YKLTY enhanced intracellular leptin sensitivity in the hypothalamus.

### Test Example 12: High-Fat Diet Intake with Oral Administration of Peptides from Spinach, Algae, and Bacteria Rubisco

The amount of food intake was measured in the same manner as in Test Example 2, except that YKLTY, YRLTY, and YRETY were used instead of YHIEPV (N=5-7). FIG. 12 shows the results. The data were analyzed by two-way analysis of variance, followed by multiple comparisons by Tukey's test. In FIG. 10, the error bars represent standard error (n=5-7).

1. A composition for improving leptin resistance or enhancing leptin sensitivity, comprising a peptide comprising the amino acid sequence YAB as an active ingredient,
wherein A is an amino acid residue having a basic side chain, and B is an amino acid residue having a nonpolar side chain or an amino acid residue having an acidic side chain.

2. The composition for improving leptin resistance or enhancing leptin sensitivity according to claim 1,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

3. A composition for improving leptin resistance or enhancing leptin sensitivity, comprising a pepsin-treated product or a pepsin-treated and pancreatin-treated product of Rubisco protein as an active ingredient.

4. A composition for suppressing food intake comprising a peptide comprising the amino acid sequence YAB as an active ingredient,
wherein A is an amino acid residue having a basic side chain, and B is an amino acid residue having a nonpolar side chain or an amino acid residue having an acidic side chain.

5. The composition for suppressing food intake according to claim 4,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

6. A composition for suppressing food intake comprising a pepsin-treated product or a pepsin-treated and pancreatin-treated product of Rubisco protein as an active ingredient.

7. A composition for anti-obesity comprising a peptide comprising the amino acid sequence YAB as an active ingredient,
wherein A is an amino acid residue having a basic side chain, and B is an amino acid residue having a nonpolar side chain or an amino acid residue having an acidic side chain.

8. The composition for anti-obesity according to claim 7,
wherein the peptide is
a peptide comprising the amino acid sequence YHIEPV (SEQ ID NO: 1),
a peptide comprising the amino acid sequence YKLTY (SEQ ID NO: 2),
a peptide comprising the amino acid sequence YRLTY (SEQ ID NO: 3), or
a peptide comprising the amino acid sequence YRETY (SEQ ID NO: 4).

9. A composition for anti-obesity comprising a pepsin-treated product or a pepsin-treated and pancreatin-treated product of Rubisco protein as an active ingredient.

10. The composition according to claim 1, which is an oral composition.

11. The composition according to claim 1, which is a pharmaceutical composition.

12. The composition according to claim 1, which is a food and drink composition.
